(19) United States
(12) Patent Application Publication
(10) Pub. No.: US 2022/0101267 A1
(43) Pub. Date: Mar. 31, 2022

(54) MEDICAL APPOINTMENT SCHEDULING SYSTEM

(71) Applicant: Kraken Medical Services, Houston, TX (US)

(72) Inventors: Michael MERGENTHALER, Cornelius, NC (US); Christopher BARNES, The Woodlands, TX (US); Russell HARRIS, Houston, TX (US); Howard STERLING, Houston, TX (US)

(21) Appl. No.: 17/548,526

(22) Filed: Dec. 11, 2021

Related U.S. Application Data
(63) Continuation of application No. 16/285,962, filed on Feb. 26, 2019, now abandoned.

(51) Int. Cl. G06Q 10/10; G16H 40/20; G16H 10/60

(52) U.S. Cl. CPC G06Q 10/1095; G16H 15/00; G16H 10/60; G16H 40/20

(57) ABSTRACT

Techniques for efficient medical appointment scheduling are described. In an example, a method of facilitating access between a patient endpoint system and a healthcare provider computer system includes: receiving, at a management computer system through a network, healthcare provider information from the healthcare provider computer; storing the healthcare provider information in a database along with a collection of healthcare provider information from a plurality of other healthcare provider computer systems, the healthcare provider information including appointment availability information; providing, from the management computer to the patient endpoint system, an interface to the database to retrieving the healthcare provider information; and providing, from the patient endpoint system to the healthcare provider computer system, a request for an appointment based on the appointment availability information retrieved by the patient endpoint system from the database.

### CROSS REFEREMCE TO RELATED APPLICATION

This application is a Continuation of pending U.S. patent application Ser. No. 16/285,962, filed Feb. 26, 2019 and entitled “MEDICAL APPOINTMENT SCHEDULING SYSTEM”, the entirety of which is incorporated by reference herein.

### BACKGROUND

Presently, the Internet plays a large role in the healthcare experience. More and more patients are choosing faster, more convenient ways to locate medical services. Some studies indicate that as much as 59% of Americans search online for healthcare information. Further studies indicate that as much as 77% of patients believe online scheduling is important.

For medical professionals, patient non-show rates can reach up to 30%. Appointments that go unfilled cost medical professionals both time and money. Further, lack of expertise and reliance on third parties make acquiring new patients a costly endeavor. Medical professionals can lose financial and operational efficiency through fragmented business systems and lack of communication vehicles other than telephones. Thus, it is desirable to provide a more efficient scheduling system for appointment scheduling for use by patients and medical professionals.

### SUMMARY

Techniques for efficient medical appointment scheduling are described. In an embodiment, a method of facilitating access between a patient endpoint system and a healthcare provider computer system includes: receiving, at a management computer system through a network, healthcare provider information from the healthcare provider computer; storing the healthcare provider information in a database along with a collection of healthcare provider information from a plurality of other healthcare provider computer systems, the healthcare provider information including appointment availability information; providing, from the management computer to the patient endpoint system, an interface to the database to retrieving the healthcare provider information; and providing, from the patient endpoint system to the healthcare provider computer system, a request for an appointment based on the appointment availability information retrieved by the patient endpoint system from the database.

Further embodiments include a non-transitory computer-readable storage medium comprising instructions that cause a computer system to carry out the above method, as well as a computer system configured to carry out the above method.

### BRIEF DESCRIPTION OF THE DRAWINGS

FIG. 1 is a block diagram depicting a computing system according to an embodiment.

FIG. 2 is a block diagram depicting a provider computer system according to an embodiment.

FIG. 3 is a block diagram depicting a management computer system according to an embodiment.

FIG. 4 is a block diagram depicting a patient endpoint system according to an embodiment.

FIG. 5 is a flow diagram depicting a method of facilitating access between a patient endpoint system and a healthcare provider computer system according to an embodiment.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Techniques for efficient medical appointment scheduling are described. Healthcare providers are currently unsure of how to market to a new generation of patients. Healthcare providers do not leverage the tools adopted in other industries to communicate and market to their changing customer base. The healthcare industry is slower to adopt to newer methods of communication that allow providers and customers to connect and share information. Accordingly, the techniques described herein provide a computer system configured to efficiently connect patients and healthcare provides for efficient appointment scheduling. The techniques increase profile and revenue through greater digital exposure. The described system provides patients doctor and practice profiles to increase interest and drive appointment traffic to healthcare professionals. The techniques maximize appointments and revenue through modernized online scheduling. Healthcare professionals are matched with new patients ready to book appointments. Canceled appointments can be filled by posting the empty slots to the system. Patients can easily locate the correct healthcare professional by using search filters, such as medical specialty, location, amenities, costs, and accepted insurance. These and further aspects are described below with respect to the drawings.

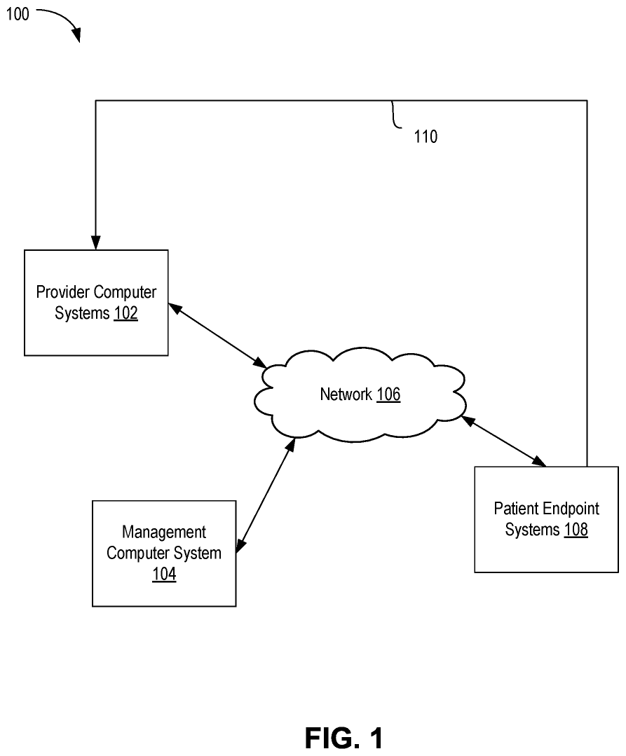

FIG. 1 is a block diagram depicting a computing system 100 according to an embodiment. The computing system 100 includes provider computer systems 102, a management computer system 104, and patient endpoint systems 108. The provider computer systems 102, the management computer system 104, and the patient endpoint systems 108 are configured for communication through a network 106. The network 106 can include one or more local area networks (LANs), wide area networks (WANs), and the like, including the public Internet.

The provider computer systems 102 can include physical computers, and/or virtual machines in a private cloud, public cloud, and/or hybrid cloud. The management computer system 104 comprises one or more physical computers and/or virtual machines in a private cloud, public could and/or hybrid cloud. The patient endpoint systems 108 comprise computers, mobile devices, and the like.

The provider computer systems 102 include computer systems operated by healthcare providers. The provider computer systems 102 execute software configured to implement appointment scheduling. The patient endpoint systems 108 are configured to provide web browser and/or application services for use by patients. The management computer system 104 executes software to maintain a database of available healthcare services of the healthcare providers and to provide an interface to the appointment scheduling systems of the healthcare providers on behalf of the patients.

In an embodiment, the patient endpoint systems 108 are configured for communication with the provider computer systems 102 through an out-of-band interface 110, such as a telephone system, e-mail system, text message system, or the like. Out-of-band interface 110 provides a direct interface between the patient endpoint systems 108 and the provider computer systems 102, rather than through the management computer system 104.

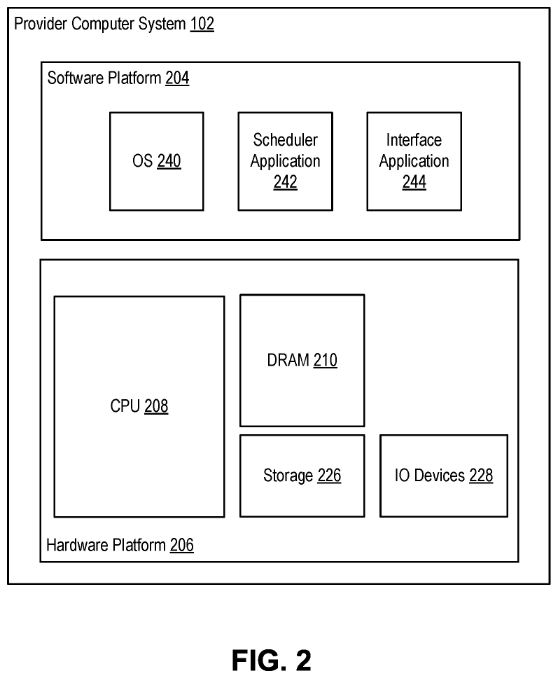

FIG. 2 is a block diagram depicting a provider computer system 102 according to an embodiment. Computer system 102 can be a physical computer or virtual machine execution in a physical computer. The provider computer system 102 includes a software platform 204 executing on a hardware platform 206. Hardware platform 206 may include conventional components of a computing device, such as a central processing unit (CPU) 208 and dynamic random access memory (DRAM) 210, as well as a storage system (storage 226), input/output devices 228, and the like. In case of a physical machine, hardware platform 206 comprises physical components. In case of a virtual machine, hardware platform 206 comprises virtual devices managed by a hypervisor or the like executing in a physical machine.

CPU 208 is configured to execute instructions, for example, executable instructions that perform one or more operations described herein and may be stored in DRAM 210 and storage 226. DRAM 210 is coupled to a memory bus of CPU 208. DRAM 210 is a device allowing information, such as executable instructions, configurations, and other data, to be stored and retrieved. DRAM 210 may include, for example, one or more random access memory (RAM) modules. Storage 226 and IO devices 228 are coupled to a peripheral bus of CPU 208. Storage 226 includes local storage devices (e.g., one or more hard disks, flash memory modules, solid state disks, and optical disks) and/or a storage interface that enables host computer 202 to communicate with one or more network data storage systems. Examples of a storage interface are a host bus adapter (HBA) that couples host computer 202 to one or more storage arrays, such as a storage area network (SAN) or a network-attached storage (NAS), as well as other network data storage systems. IO devices 228 include various network interfaces and the like.

Software platform 204 includes an operating system (OS) 240 and a scheduler application 242. In some embodiments, software platform 204 includes an interface application 244. OS 240 executes directly on hardware platform 202. OS 240 can be any commodity operating system known in the art, such as such as Linux®, Microsoft Windows®, Mac OS®, or the like. Scheduler application 242 is configured to manage appointments for healthcare providers to provide services to patients. Scheduler application 242 can be an off-the-shelf application purchased by the healthcare provider(s) for the purpose of handling patient appointments. Alternatively, scheduler application 242 can be a proprietary system developed for the healthcare providers for the purpose of scheduling patient appointments.

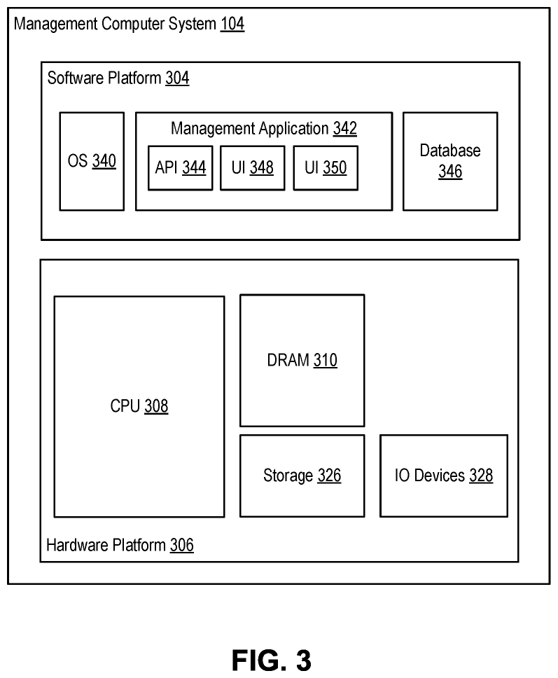

FIG. 3 is a block diagram depicting the management computer system 104 according to an embodiment. Computer system 104 can be a physical computer or virtual machine execution in a physical computer. The management computer system 104 includes a software platform 304 executing on a hardware platform 306. Hardware platform 306 may include conventional components of a computing device, such as CPU 308 and DRAM 310, as well as a storage system (storage 326), input/output devices 328, and the like. In case of a physical machine, hardware platform 306 comprises physical components. In case of a virtual machine, hardware platform 306 comprises virtual devices managed by a hypervisor or the like executing in a physical machine.

CPU 308 is configured to execute instructions, for example, executable instructions that perform one or more operations described herein and may be stored in DRAM 310 and storage 326. DRAM 310 is coupled to a memory bus of CPU 308. DRAM 310 is a device allowing information, such as executable instructions, configurations, and other data, to be stored and retrieved. DRAM 310 may include, for example, one or more random access memory (RAM) modules. Storage 326 and IO devices 328 are coupled to a peripheral bus of CPU 308. Storage 326 includes local storage devices (e.g., one or more hard disks, flash memory modules, solid state disks, and optical disks) and/or a storage interface that enables host computer 302 to communicate with one or more network data storage systems. Examples of a storage interface are a host bus adapter (HBA) that couples host computer 302 to one or more storage arrays, such as a storage area network (SAN) or a network-attached storage (NAS), as well as other network data storage systems. IO devices 328 include various network interfaces and the like.

Software platform 304 includes an OS 340 and a management application 342. OS 340 executes directly on hardware platform 302. OS 340 can be any commodity operating system known in the art, such as such as Linux®, Microsoft Windows®, Mac OS®, or the like. Management application 342 is configured to provide an application programming interface (API) 344 to receive scheduling information from provider computer systems 102. In an embodiment, the scheduler application 242 is configured to invoke the API 344 to provide the scheduling information to the management application 342. In another embodiment, the interface application 244 provides an interface between the scheduler application 242 and API 344 of the management application 342. In either embodiment, the API 344 allows the healthcare provide to deliver scheduling information and/or other associated information to the management application over time, including information describing the services provided by the healthcare providers, the healthcare providers themselves, location(s) where the services are provided, contact information, and appointment availability. Management application 342 is configured to manage a database 346. The database 346 stores the information received from the healthcare providers through the API 344.

Management application 342 also includes a provider user interface (UI) 348 and a patient UI 350. The provider UI 348 can be accessed through a web browser or a dedicated application executing on any computing device. The provider UI 348 allows healthcare providers to enroll in services provided by the operator of the management computer system 104. The patient UI 350 can be accessed through a web browser or a dedicated application executing on a patient endpoint system 108. The patient UI 350 allows patients to access information collected from healthcare providers, including appointment availability information. The management application 342 is configured to allow patients to query the database 346 to obtain information related to healthcare providers through the patient UI 350. The management application 342 is also configured to return information to the scheduler application 242 or the interface application 244 (if present). The return information can include acknowledgements and the like indicating receipt of the information provided by the healthcare providers. In an embodiment, the return information can also include booking information describing requests for appointments and contact information of patients requesting the appointments.

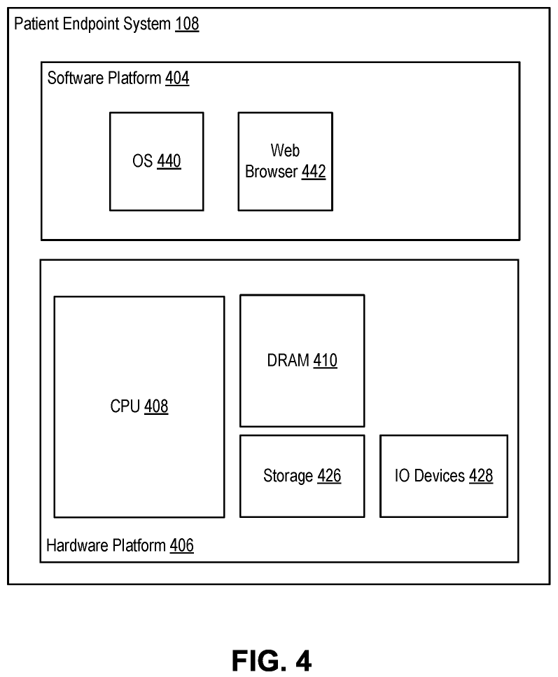

FIG. 4 is a block diagram depicting a patient endpoint system 108 according to an embodiment. Patient endpoint system 108 can be a computer, mobile device, or the like operated by patients. The patient endpoint system 108 includes a software platform 404 executing on a hardware platform 406. Hardware platform 406 may include conventional components of a computing device, such as CPU 408 and DRAM 410, as well as a storage system (storage 426), input/output devices 428, and the like. The input/output devices 428 can include a display that provides a graphical user interface (GUI) 429 to the user.

CPU 408 is configured to execute instructions, for example, executable instructions that perform one or more operations described herein and may be stored in DRAM 410 and storage 426. DRAM 410 is coupled to a memory bus of CPU 408. DRAM 410 is a device allowing information, such as executable instructions, configurations, and other data, to be stored and retrieved. DRAM 4310 may include, for example, one or more random access memory (RAM) modules. Storage 426 and IO devices 428 are coupled to a peripheral bus of CPU 308. Storage 426 includes local storage devices (e.g., one or more hard disks, flash memory modules, solid state disks, and optical disks) IO devices 428 include various network interfaces and the like.

Software platform 404 includes an OS 440 and a web browser 442. OS 440 executes directly on hardware platform 402. OS 440 can be any commodity operating system known in the art, such as such as Linux®, Microsoft Windows®, Mac OS®, or the like. Web browser 442 can be any application configured to access the patient UI 350 through the network 106.

Referring to FIGS. 1-4, the management application 342 provides a service to patients and healthcare providers that enables more options and opportunities for patients to access information that will improve decisions on healthcare. The interaction between the management application 342, the scheduler application 242, and the patient endpoint device 108 provides for improved technology that provides the following benefits. The management application 342 reduces time to schedule appointments by provide an API 344 between the scheduler application 242 and the database 346. The management application 342 identifies and presents appointment opportunities in real time based on location of consumer and healthcare provider and the provider's specialty area of practice. The management application 342 consolidates information for healthcare consumers by (1) collecting fee information from providers and presents common fees based on location and specialties; (2) allows consumers to make purchase decisions based on a healthcare provider's educational background and gender; and 3) allows consumers to make healthcare purchasing decisions based on other services/amenities the provider has, such as parking, on-site pharmacy, and the like. Patients can search for healthcare providers and have the results sorted by distance from a location, price, first available appointment opportunity, education/medical school, other amenities (e.g., pharmacy), and the like.

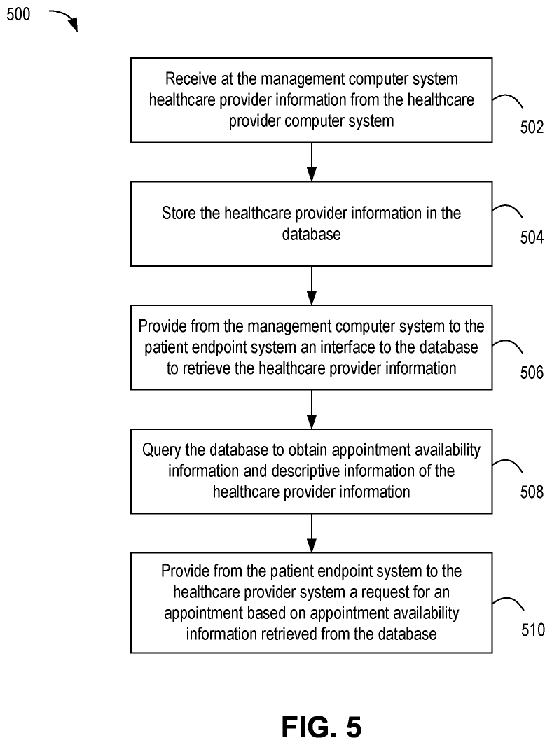

FIG. 5 is a flow diagram depicting a method 500 of facilitating access between a patient endpoint system 108 and a healthcare provider computer system 102 according to an embodiment. The method 500 begins at step 502, where the management computer system 104 receives healthcare provider information from the healthcare provide computer system 102. The healthcare provider information includes appointment availability information and descriptive information. The descriptive information can include various data, such as data describing healthcare providers, services, and facilities. At step 504, the management computer system 104 stores the healthcare provider information in a database 346 along with a collection of healthcare provider information from multiple other healthcare provide computer systems.

At step 506, the management computer system 104 provides a UI 350 to the patient endpoint system 108 to provide an interface for querying the database 346 to obtain the healthcare information. At step 508, the patient endpoint system 108 queries the database 346 to obtain appointment availability information and descriptive information of the healthcare provider information. At step 510, the patient endpoint system 108 provides a request for an appointment to the healthcare provider system 102 based on the appointment availability information retrieved from the database 346. The appointment request can be delivered through different mechanisms depending on capabilities of the management computer system 104, the healthcare provider computer system 102, and the patient endpoint system 108. In an embodiment, the patient computer system 108 provides the appointment request through the management computer system 104. In another embodiment, the patient computer system 108 provides the appointment request through the out-of-band interface 110, such as by a telephone call, e-mail, text message, or the like.

The various embodiments described herein may employ various computer-implemented operations involving data stored in computer systems. For example, these operations may require physical manipulation of physical quantities—usually, though not necessarily, these quantities may take the form of electrical or magnetic signals, where they or representations of them are capable of being stored, transferred, combined, compared, or otherwise manipulated. Further, such manipulations are often referred to in terms, such as producing, identifying, determining, or comparing. Any operations described herein that form part of one or more embodiments of the invention may be useful machine operations. In addition, one or more embodiments of the invention also relate to a device or an apparatus for performing these operations. The apparatus may be specially constructed for specific required purposes, or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

The various embodiments described herein may be practiced with other computer system configurations including hand-held devices, microprocessor systems, microprocessor-based or programmable consumer electronics, minicomputers, mainframe computers, and the like.

One or more embodiments of the present invention may be implemented as one or more computer programs or as one or more computer program modules embodied in one or more computer readable media. The term computer readable medium refers to any data storage device that can store data which can thereafter be input to a computer system—computer readable media may be based on any existing or subsequently developed technology for embodying computer programs in a manner that enables them to be read by a computer. Examples of a computer readable medium include a hard drive, network attached storage (NAS), read-only memory, random-access memory (e.g., a flash memory device), a CD (Compact Discs)—CD-ROM, a CD-R, or a CD-RW, a DVD (Digital Versatile Disc), a magnetic tape, and other optical and non-optical data storage devices. The computer readable medium can also be distributed over a network coupled computer system so that the computer readable code is stored and executed in a distributed fashion.

Although one or more embodiments of the present invention have been described in some detail for clarity of understanding, it will be apparent that certain changes and modifications may be made within the scope of the claims. Accordingly, the described embodiments are to be considered as illustrative and not restrictive, and the scope of the claims is not to be limited to details given herein, but may be modified within the scope and equivalents of the claims. In the claims, elements and/or steps do not imply any particular order of operation, unless explicitly stated in the claims.

Virtualization systems in accordance with the various embodiments may be implemented as hosted embodiments, non-hosted embodiments or as embodiments that tend to blur distinctions between the two, are all envisioned. Furthermore, various virtualization operations may be wholly or partially implemented in hardware. For example, a hardware implementation may employ a look-up table for modification of storage access requests to secure non-disk data.

Many variations, modifications, additions, and improvements are possible, regardless the degree of virtualization. The virtualization software can therefore include components of a host, console, or guest operating system that performs virtualization functions. Plural instances may be provided for components, operations or structures described herein as a single instance. Boundaries between various components, operations and data stores are somewhat arbitrary, and particular operations are illustrated in the context of specific illustrative configurations. Other allocations of functionality are envisioned and may fall within the scope of the invention(s). In general, structures and functionality presented as separate components in exemplary configurations may be implemented as a combined structure or component. Similarly, structures and functionality presented as a single component may be implemented as separate components. These and other variations, modifications, additions, and improvements may fall within the scope of the appended claim(s).

What is claimed is:

1. A method of facilitating access between a patient endpoint system and a healthcare provider computer system, comprising:
receiving, at a management computer system through a network, healthcare provider information from at least two healthcare provider computer systems including appointment availability information;
storing the healthcare provider information in a database along with a collection of healthcare provider information from a plurality of other healthcare provider computer systems, the healthcare provider information including appointment availability information;
providing, from the management computer to the patient endpoint system, an interface to the database to retrieving the healthcare provider information;
providing, from the patient endpoint system to the healthcare provider computer system, a request for an appointment based on the appointment availability information retrieved by the patient endpoint system from the database prior to the healthcare provider computer system receiving at the management computer system the healthcare provider information; and
generating in real time a plurality of available appointment times and present the plurality of available appointment times to the patient endpoint system and receiving a user selection of a presented appointment time.

2. The method of claim 1, wherein the management computer system includes an application programming interface (API) therein configured to receive the healthcare provider information from a scheduler application executing in the healthcare provider computer.

3. The method of claim 2, wherein the healthcare provider computer includes an interface application configured to provide an interface between the API and the scheduler application.

4. The method of claim 1, wherein the healthcare provider information includes descriptive information describing healthcare providers, services, and facilities, and wherein the patient endpoint system is configured to retrieve the descriptive information from the database.

5. The method of claim 1, wherein the step of providing the request for the appointment comprises:
sending the request for the appointment to the healthcare provider computer system through the management computer system.

6. The method of claim 1, wherein the step of providing the request for the appointment comprises:
sending the request for the appointment to the healthcare provider computer system through an out-of-band interface between the patient endpoint system and the healthcare provider computer system.

7. The method of claim 6, wherein the out-of-band interface comprises a telephone interface, an e-mail interface, or a text message interface.

8. A non-transitory computer readable medium having instructions stored thereon configured to cause a processor to execute a method of facilitating access between a patient endpoint system and a healthcare provider computer system, comprising:
receiving, at a management computer system through a network, healthcare provider information from at least two healthcare provider computer systems including appointment availability information;
storing the healthcare provider information in a database along with a collection of healthcare provider information from a plurality of other healthcare provider computer systems, the healthcare provider information including appointment availability information;
providing, from the management computer to the patient endpoint system, an interface to the database to retrieving the healthcare provider information;
providing, from the patient endpoint system to the healthcare provider computer system, a request for an appointment based on the appointment availability information retrieved by the patient endpoint system from the database prior to the healthcare provider computer system receiving at the management computer system the healthcare provider information; and
generating in real time a plurality of available appointment times and present the plurality of available appointment times to the patient endpoint system and receiving a user selection of a presented appointment time.

9. The non-transitory computer readable medium of claim 8, wherein the management computer system includes an application programming interface (API) therein configured to receive the healthcare provider information from a scheduler application executing in the healthcare provider computer.

10. The non-transitory computer readable medium of claim 9, wherein the healthcare provider computer includes an interface application configured to provide an interface between the API and the scheduler application.

11. The non-transitory computer readable medium of claim 8, wherein the healthcare provider information includes descriptive information describing healthcare providers, services, and facilities, and wherein the patient endpoint system is configured to retrieve the descriptive information from the database.

12. The non-transitory computer readable medium of claim 8, wherein the step of providing the request for the appointment comprises:
sending the request for the appointment to the healthcare provider computer system through the management computer system.

13. The non-transitory computer readable medium of claim 8, wherein the step of providing the request for the appointment comprises:
sending the request for the appointment to the healthcare provider computer system through an out-of-band interface between the patient endpoint system and the healthcare provider computer system.

14. The non-transitory computer readable medium of claim 13, wherein the out-of-band interface comprises a telephone interface, an e-mail interface, or a text message interface.

15. A computing system, comprising:
a healthcare provide computer system, a management computer system, and a patient endpoint system;
wherein the management computer system is configured to facilitate access between the patient endpoint system and the healthcare provider computer system by:
receiving, through a network, healthcare provider information from at least two healthcare provider computer systems including appointment availability information;
storing the healthcare provider information in a database along with a collection of healthcare provider information from a plurality of other healthcare provider computer systems, the healthcare provider information including appointment availability information;
providing, to the patient endpoint system, an interface to the database to retrieving the healthcare provider information;
wherein the patient endpoint system is configured to provide to the healthcare provider computer system, a request for an appointment based on the appointment availability information retrieved by the patient endpoint system from the database prior to the healthcare provider computer system receiving at the management computer system the healthcare provider information; and
generating in real time a plurality of available appointment times and present the plurality of available appointment times to the patient endpoint system and receiving a user selection of a presented appointment time.

16. The computing system of claim 15, wherein the management computer system includes an application programming interface (API) therein configured to receive the healthcare provider information from a scheduler application executing in the healthcare provider computer.

17. The computing system of claim 16, wherein the healthcare provider computer includes an interface application configured to provide an interface between the API and the scheduler application.

18. The computing system of claim 15, wherein the healthcare provider information includes descriptive information describing healthcare providers, services, and facilities, and wherein the patient endpoint system is configured to retrieve the descriptive information from the database.

19. The computing system of claim 15, wherein the step of providing the request for the appointment comprises:
sending the request for the appointment to the healthcare provider computer system through the management computer system.

20. The computing system of claim 15, wherein the step of providing the request for the appointment comprises:
sending the request for the appointment to the healthcare provider computer system through an out-of-band interface between the patient endpoint system and the healthcare provider computer system.
